(19) United States
(12) Patent Application Publication
(10) Pub. No.: US 2017/0028116 A1
(43) Pub. Date: Feb. 2, 2017

(54) AORTIC CATHETER

(71) Applicant: MEDIZINISCHE UNIVERSITAET WIEN, Vienna (AT)

(72) Inventors: Fritz Sterz, Vienna (AT); Andreas Janata, Vienna (AT); Wolfgang Weihs, Blumau-Neurisshof (AT); Christoph Weiser, Korneuburg (AT); Michael Holzer, Vienna (AT); Wilhelm Behringer, Vienna (AT)

(21) Appl. No.: 15/106,326

(22) PCT Filed: Dec. 19, 2014
(86) PCT No.: PCT/AT2014/050310

§ 371 Date: Jun. 20, 2016

(30) Foreign Application Priority Data
Dec. 19, 2013 (AT) 50837/2013

(51) Int. Cl. A61M 1/10; A61B 90/00; A61F 7/12; A61M 25/01; A61M 1/12; A61M 1/36

(52) U.S. Cl. CPC A61M 1/1072; A61M 1/125; A61M 1/3659; A61F 7/12; A61M 25/0108; A61B 90/39; A61F 2007/126; A61F 2007/0063

(57) ABSTRACT

The invention relates to an aortic catheter (1) for insertion into the aorta (A), having a flexible tube (2), two occlusion balloons (3, 4), which are spaced part from one another and each of which is connected to a supply line (5, 6) for supplying a pumping medium (7) for inflating the occlusion balloons (3, 4), having at least one opening (8) arranged between the occlusion balloons (3, 4) in the tube (2) for supplying a cooling medium (9), which at least one opening (8) is connected to a first cooling medium supply line (10) running in the tube (2), and also relates to a resuscitation set (22) with such an aortic catheter (1), and a distal port (13) for supplying a cooling medium (9) in the direction of cerebral vessels is disposed in the tube (2), which distal port (13) is connected to a second cooling medium supply line (14) running in the tube (2).

The invention relates to an aortic catheter for insertion into the aorta, having a flexible tube, two occlusion balloons, which are spaced a distance apart from one another and each of which is connected to a supply line for supplying a pumping medium for inflation of the occlusion balloons, and having at least one opening arranged between the occlusion balloons in the tube for supplying a cooling medium, which at least one opening being connected to a first cooling medium supply line running in the tube.

The invention also relates to a resuscitation set having such an aortic catheter, a tank for the cooling medium, a pump for pumping the cooling medium into the cooling medium supply lines and, if necessary, the cooling medium supply to the aortic catheter, a pump for operating the intra-aortic balloon pump and a heart monitor.

The chance of survival of patients after cardiac arrest, trauma, cardiogenic shock and/or a stroke can be increased significantly by rapidly lowering their body temperature. By inducing so-called hypothermia, the oxygen consumption by the patient's brain is reduced and various cellular decomposition processes that can result in irreparable neurological damage are retarded. In addition to noninvasive induction of hypothermia, for example, by means of cooling blankets or the like, there are also invasive methods of inducing hypothermia, by administering a cooling liquid to the bloodstream through a catheter, so that the body temperature is lowered very rapidly.

US 2008/0221553 A1, for example, describes an aortic catheter having two occlusion balloons for infusion of a cooling liquid through openings between the occlusion balloons on the catheter. By using such a catheter, important organs can be cooled by means of the arteries supplying blood to these organs.

A device for cerebral resuscitation using a catheter for injecting a cooling fluid is already known from U.S. Pat. No. 5,149,321, for example. Thereby, the cooling liquid is introduced through the carotid artery and therefore the temperature of the patient's brain is lowered.

Finally, US 2010/0121273 A1 describes an invasive method of inducing hypothermia with the help of a catheter, through which the cooling fluid, in particular a saline solution, is infused. No details were disclosed about the catheter.

Since the patient must be connected to a heart-lung machine after hypothermia has been induced in a cardiac arrest case and then must be disconnected from the heart-lung machine after successful resuscitation, it is usually necessary to replace the catheter used to induce hypothermia by intra-aortic balloon pumps, but this is complicated and requires time, which is usually not available in such emergencies. Furthermore, simultaneous induction of hypothermia in cerebral regions and peripheral regions of a patient is impossible with traditional catheters.

The object of the present invention therefore consists of creating such an aortic catheter and a resuscitation set equipped with such a catheter, with which a patient can receive optimal care with the least possible effort for the attending personnel. Disadvantages of known catheters for inducing hypothermia should be avoided or at least reduced.

The object according to the invention is achieved by an aortic catheter, with which an intra-aortic balloon pump is situated between the occlusion balloons and is connected to another supply line, and a distal port is provided in the tube for supplying a cooling medium in the direction of the cerebral vessels, this distal port being connected to a second cooling medium supply line that runs in the tube. The aortic catheter according to the invention permits cooling of the most important organ such as kidneys, stomach, intestine, pancreas, liver, spleen and spinal cord as well as cooling the patient's brain, so that the patient's chance of survival can be greatly increased. In addition, the aortic catheter is equipped with an intra-aortic balloon pump and a connection to a heart-lung machine. Thus it is possible to proceed with maintaining the patient's circulation with the help of the heart-lung machine without replacing the catheter during or after the hypothermia induction. The aortic catheter according to the invention is thus characterized by a variety of functions and/or a combination of a catheter for inducing hypothermia and maintaining hypothermia, for taking over the lung function, as a cardiopulmonary bypass and as an intra-aortic balloon pump. The aortic catheter according to the invention thus optimally supports the patient and contributes toward an increased probability of completely recovery. The first and second cooling medium supply lines may also be formed by a joint cooling medium supply line.

The ratio of the cross-section of the first cooling medium supply line to the cross-section of the second cooling medium supply line is advantageously between 1:1.75 and 1:2.25, in particular 1:2. Due to such cross-section ratios, the supply of the brain and the organs with the cooling fluid in a ratio of approximately 70:30% is achieved, i.e., more cooling fluid for the brain than for the organs and the spinal cord is introduced through the catheter.

Optimal cooling of the organs and the spinal cord by means of the supplying arteries can be achieved when multiple openings are arranged between the occlusion balloons in the tube, these openings having a diameter between 1 mm and 2 mm. When inserting the catheter, care should be taken to ensure that the distal occlusion balloon is placed beneath the carotid artery outlets and the second occlusion balloon is placed beneath the arteries supplying the kidneys, so that the supply of cooling fluid to the most important organs is ensured.

The two occlusion balloons are preferably arranged at a distance of 20 cm to 30 cm from one another. This distance seems to be suitable for adult patients. With normal aorta diameters between 2.5 cm and 3 cm in adults, these dimensions are optimal for the aortic occlusion balloons on the catheter.

The aortic catheter can be used in adults if the tube has an outside diameter of 5 mm to 10 mm between the occlusion balloons.

To enable additional tests or intervention procedures without changing the aortic catheter, a lumen connected to a distal opening is provided in the tube for insertion of instruments. For example, it is possible to perform an examination with a cardiac catheter in this way after stabilizing the patient but without changing the catheter as long as a suitably tight introducer sheath is provided. The second supply line for cooling medium to the distal port may also be used as a lumen for insertion of instruments if a corresponding introducer sheath is provided.

If a cooling device which is connected to a coolant supply and a coolant drain is provided in the intra-aortic balloon pump, a maintenance cooling of the patient can be carried out. The cooling device in the intra-aortic balloon pump may in the simplest case be established by a connection between a coolant supply and a coolant drain through a corresponding sheath wall in the balloon pump. As an alternative to this, the cooling medium may also be guided in meandering or spiral-shaped cooling channels inside the balloon pump and used for cooling the circulating blood by the heat exchanger principle.

At least one temperature sensor may be provided in the aortic catheter, so that the patient's core temperature can be measured. The hypothermia induction can be controlled on the basis of the temperature thereby determined.

At least one temperature sensor is preferably arranged on the distal end of the tube. The connection between the temperature sensor and an external measurement device may be hardwired or may also be achieved as a wireless radio connection.

If markers made of radiopaque material are provided on the tube, the correct placement of the aortic catheter can be monitored by using an X-ray device. In the simplest case, rings of metal material may be inserted into the aortic catheter, so that, in an emergency, the position of the catheter is indicated to the medical personnel performing X-ray monitoring.

The cooling medium for inducing hypothermia may be formed by a crystalloid solution. Crystalloid solutions or electrolyte solutions are known for infusion therapies for equalizing or covering a patient's fluid needs. In the simplest case, the electrolyte solution is formed by an isotonic saline solution.

The pumping medium for the occlusion balloons may consist of helium, for example. Helium is particularly suitable because it does not cause any damage in the event of leakage of the occlusion balloons. As an alternative to this gas, use of physiological saline solution for inflating the occlusion balloons is also conceivable.

The task according to the invention is also achieved by a resuscitation set as defined above with an aortic catheter as described above, wherein a control unit, which is connected to the heart monitor and the pump for pumping the cooling medium and the pump for operating the intra-aortic balloon pump, is provided. Such a resuscitation set, which contains all the instruments required for the treatment of the patient, may be used in ambulance vehicles as well as in outpatient clinics of hospitals. To enable immediate use, it is necessary to ensure that the cooling medium is kept at the proper temperature. This may be achieved, for example, by using the corresponding cooling units. The resuscitation set should therefore be connected to a corresponding voltage source and should also preferably be provided with batteries, which maintain the operation of the components even without a connection to the power supply system.

The components are preferably accommodated in a carrying case or the like so that rapid and simple transport to the patient is made possible. Such a carrying case or the like can be carried along in an emergency vehicle, for example, or may be kept on hand in an emergency medical ward of a hospital. Since the aortic catheter is a disposable product, multiple aortic catheters may be provided in suitable sterile packages.

For the possibility of facilitated transport, the carrying case or the like preferably has wheels and a handlebar, as is customary with travel baggage items.

Alternatively or in addition to that, carrying straps or shoulder straps may also be provided on the carrying case or the like, so that the carrying case or the like can be carried conveniently over the shoulder or on the back.

A defibrillator, which may also be combined with a monitor, is preferably also provided.

In addition, a cardiac catheter may also be provided in the resuscitation set. This cardiac catheter can be inserted into the aortic catheter over the existing continuous lumen. In this way, the cardiac catheter examination can be performed without replacing the catheter.

The control unit of the resuscitation set may have an input for connecting a temperature sensor. Therefore, for temperature control and monitoring of the hyperthermia, the patient's core temperature can also be measured at a suitable location, for example, in the esophagus, and included in the control.

For optimal operation of the resuscitation set, the control unit preferably has an input/output unit. This input/output unit consists of suitable operating elements and output elements or a display screen and/or a touchscreen, which combines the input and output functions.

The invention will now be described in greater detail on the basis of the accompanying drawings, in which:

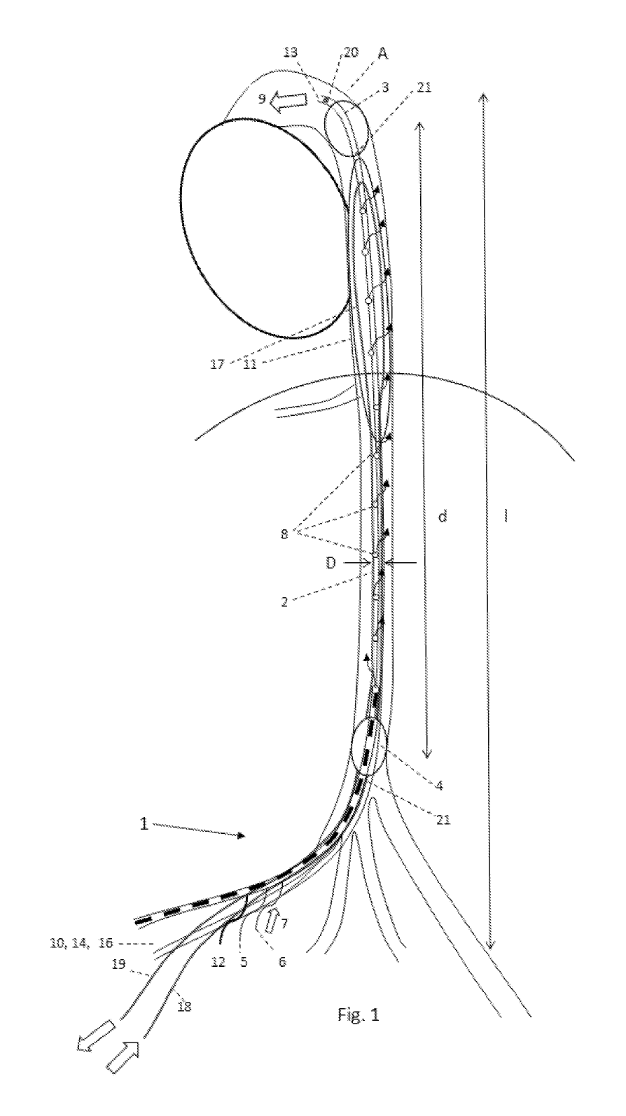

FIG. 1 shows a basic diagram of the aortic catheter according to the invention;

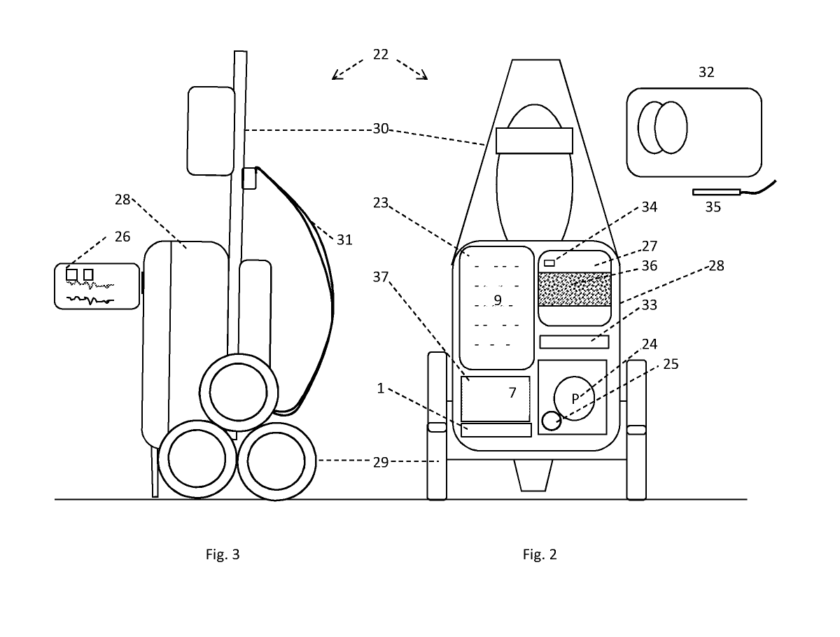

FIG. 2 shows a partial sectional diagram of a resuscitation set according to the present invention;

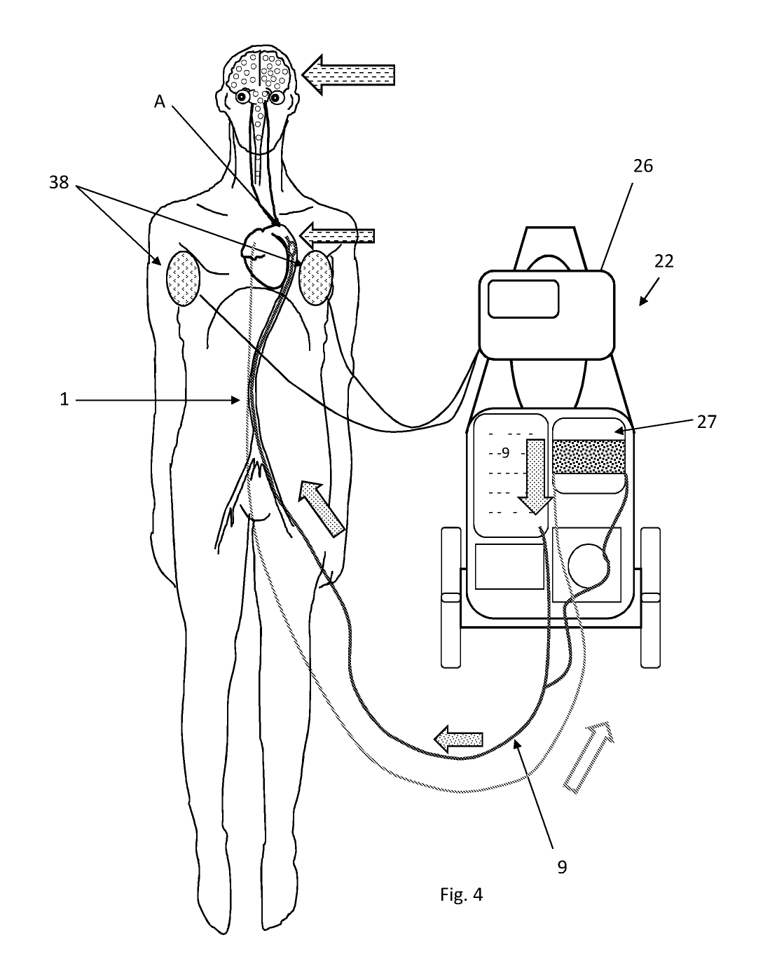

FIG. 3 shows a side view of a resuscitation set; and

FIG. 4 shows another basic diagram of a resuscitation set being used on a patient.

FIG. 1 shows a basic diagram of an aortic catheter according to the invention 1, which is situated in a patient's aorta A. The aortic catheter 1 comprises a flexible tube 2 and a distal occlusion balloon 3 as well as a proximal occlusion balloon 4, which are arranged at a distance d of preferably 20 cm to 30 cm from one another. The total length 1 of the aortic catheter 1 is between 50 cm and 60 cm to permit use in adult patients. The diameter D of the tube 2 is preferably 5 mm to 10 mm between the occlusion balloons 3, 4. The occlusion balloon 3 is supplied with a corresponding pumping medium 7, for example, helium, through a supply line 5, and the proximal occlusion balloon 4 is supplied with the pumping medium 7 through a supply line 6, and closure of the aorta A is induced after the aortic catheter 1 has been positioned. Openings 8 for supplying a cooling medium 9 are arranged between the occlusion balloons 3, 4 in the tube 2 of the aortic catheter 1. The openings 8 in the tube are connected to a first cooling medium supply line 10. In addition, an intra-aortic balloon pump 11 with a supply line 12 is arranged between the occlusion balloons 3, 4. The patient's circulation can be maintained with the assistance of a heart-lung machine (not shown) by way of the intra-aortic balloon pump 11. In addition, a distal port 13 is arranged on the distal end of the aortic catheter 1 and is connected to a second cooling medium supply line 14, through which the cooling medium 9 can be introduced. This cooling medium is injected through the aortic catheter 1 into the patient's brain, where it induces hypothermia. To be able to introduce most of the cooling medium through the distal port 13, whereas only approximately 30% of the cooling medium 9 is introduced through the openings 8, the ratio of the cross-section of the first cooling medium supply line 10 to the cross-section of the second cooling medium supply line 14 is between 1:1.75 and 1:2.25, in particular 1:2. The cerebral regions of the patient are thus supplied with more cooling medium 9 than are the internal organs or the spinal cord. The first cooling medium supply line 10 and the second cooling medium supply line 14 can also be realized by a shared cooling medium supply (not shown), wherein the quantitative division of the cooling medium 9 can be adjusted through the size of the distal port 13 and the openings 8. Instruments such as a cardiac catheter can be inserted through an additional lumen 16, which extends from the proximal end to the distal end of the aortic catheter 1 if a suitably tight introducer sheath is arranged in the lumen 16. The second cooling medium supply line 14, which is connected to the distal port 13, may of course also be used with a corresponding introducer sheath for introducing instruments. A maintenance cooling can be achieved with a heart-lung machine during the treatment of the patient by means of a cooling device 17 in the intra-aortic balloon pump 11, which is connected to a coolant supply line 18 and a coolant drain line 19. The cooling device 17 acts as a heat exchanger and delivers the cold of the cooling medium to the circulating blood and thus increases the patient's chances of survival by inducing hypothermia. A temperature sensor 20 may preferably be provided at the distal end of the aortic catheter 1 to detect the patient's core temperature. Corresponding markers 21 of radiopaque material can indicate the correct position of the aortic catheter 1 under an X-ray device.

The aortic catheter 1 according to the invention serves as a so-called flush catheter for invasive induction of hypothermia, while also serving as a bypass catheter for maintaining pulmonary function and as an intra-aortic balloon pump 11 for use in combination with a heart-lung machine, permitting a cardiac catheter examination or the like and endovascular cooling by means of the cooling device 17 in the intra-aortic balloon pump 11. Such a multifunction catheter can serve to substantially increase the chance of survival of a cardiac arrest patient.

FIG. 2 shows one possible embodiment of a resuscitation set 22 which has an aortic catheter 1, which was described above with reference to FIG. 1, a tank 23 for the cooling medium 9, a pump 24 for pumping the cooling medium 9 into the cooling medium supply lines 10, 14 and the cooling medium supply 19 of the aortic catheter 1 as well as a pump 25 for operating the intra-aortic balloon pump 11 and a container 37 for the pumping medium 7 for pumping up the occlusion balloons 3, 4. In addition, a heart monitor 26 is provided and may also be combined with a defibrillator 32. A control unit 27 controls the corresponding components, such as the pump 24, for pumping the cooling medium 8, the pump 25 for operating the intra-aortic balloon pump 11, etc. The components of the resuscitation set 22 are preferably arranged in a carrying case 28 or the like, which has wheels 29 and a handlebar 30 for facilitating easy transport. In addition, the carrying case 28 may also have carrying straps 31 or the like. An external temperature sensor 35 may be provided, for example, in a respiration tube (not shown) by means of an input 34 on the control unit 27. An input/output unit 36 is preferably provided for operating the control unit 27.

FIG. 4 shows a basic diagram of use of the resuscitation set on a patient, wherein the aortic catheter 1 was positioned by means of the femoral artery and connected by means of corresponding lines, which are in the carrying case 28 of the resuscitation set 22. Hypothermia can be induced rapidly through the aortic catheter 1 and a significant increase in the patient's chance of survival can be achieved in this way. Cardiac activity is determined by means of suitable electrodes 38, which are connected to the heart monitor 26, with the signals being forwarded to the control unit 27.

1. An aortic catheter for insertion into the aorta (A), comprising:
a flexible tube,
two occlusion balloons spaced a distance apart from one another, each connected to a supply line for supplying a pumping medium for inflating the occlusion balloons and
the flexible tube having at least one opening arranged between the occlusion balloons for supplying a cooling medium, the at least one opening being connected to a first cooling medium supply line running in the tube, wherein
an intra-aortic balloon pump, which is arranged between the occlusion balloons, is connected to another supply line, and
a distal port for supplying a cooling medium in direction of an cerebral vessels, is arranged in the tube, said distal port being connected to a second cooling medium supply line running in the tube.

2. The aortic catheter according to claim 1, wherein a ratio of a cross-section of the first cooling medium supply line to a cross-section of the second cooling medium supply line is between 1:1.75 and 1:2.25.

3. The aortic catheter according to claim 1, wherein multiple openings are arranged in the tube between the occlusion balloons, said openings having a diameter between 1 mm and 2 mm.

4. The aortic catheter according to claim 1, wherein the two occlusion balloons are arranged at a distance of 20 cm to 30 cm from one another.

5. The aortic catheter according to claim 1, wherein the tube has an outside diameter of 5 mm to 10 mm between the occlusion balloons.

6. The aortic catheter according to claim 1, wherein a lumen connected to a distal opening for inserting instruments is arranged in the tube.

7. The aortic catheter according to claim 1, wherein a cooling device which is provided in the intra-aortic balloon pump is connected to a coolant supply and a coolant drain line.

8. The aortic catheter according to claim 1, wherein at least one temperature sensor is provided.

9. The aortic catheter according to claim 8, wherein said at least one temperature sensor is arranged on a distal end of the tube.

10. The aortic catheter according to claim 1, wherein markers made of a radiopaque material are arranged on the tube.

11. The aortic catheter according to claim 1, wherein the cooling medium is formed by a crystalloid solution.

12. The aortic catheter according to claim 1, wherein the pumping medium is formed by helium.

13. A resuscitation set comprising:
an aortic catheter according to claim 1,
a tank for the cooling medium,
a pump for pumping the cooling medium into the cooling medium supply lines and,
optionally,
a cooling medium supply of the aortic catheter,
a pump for operating the intra-aortic balloon pump, and
a heart monitor, wherein a control unit is provided which is connected to the heart monitor and the pump for pumping the cooling medium and to the pump for operating the intra-aortic balloon pump.

14. The resuscitation set according to claim 13, wherein the components are arranged in a carrying case.

15. The resuscitation set according to claim 14, wherein the carrying case has wheels and a handlebar.

16. The resuscitation set according to claim 14, wherein the carrying case has carrying straps.

17. The resuscitation set according to claims 13, wherein a defibrillator is provided.

18. The resuscitation set according to claims 13, wherein a cardiac catheter is provided.

19. The resuscitation set according to claims 13, wherein the control unit has an input for connecting a temperature sensor.

20. The resuscitation set according to claims 13, wherein the control unit has an input/output unit.

21. The aortic catheter according to claim 2, wherein the ratio of the cross-section of the first cooling medium supply line to the cross-section of the second cooling medium supply line is between 1:2.
